(19) United States
(12) Patent Application Publication
(10) Pub. No.: US 2012/0316517 A1
(43) Pub. Date: Dec. 13, 2012

(54) WOUND DRESSING CONTAINING FOAM AND OINTMENT BASE AND SWELLING AGENT FOR NEGATIVE PRESSURE THERAPY

(76) Inventors: Pierre Croizat, Herbrechtingen (DE); Axel Eckstein, Heidenheim (DE); Antonia Weis, St. Augustin (DE)

(21) Appl. No.: 13/491,236

(22) Filed: Jun. 7, 2012

Related U.S. Application Data
(60) Provisional application No. 61/494,949, filed on Jun. 9, 2011.

(30) Foreign Application Priority Data
Jun. 9, 2011 (EP) 11004724

(51) Int. Cl. A61K 9/70; A61M 35/00; A61M 1/00; A61P 17/02

(52) U.S. Cl. 604/304; 424/445

(57) ABSTRACT

The invention relates to a device for the negative pressure treatment of wounds comprising (a) a covering material for providing a wound area with an air-tight seal; (b) optionally means for connecting a negative pressure source; and (c) a wound dressing containing (c1) an open-celled foamed material which is wetted with (c2) an ointment base and (c3) a swelling material, and also a process for producing a corresponding wound dressing. In addition, the invention relates to the use of an open-celled foamed material, which is wetted with an ointment base, for application as a wound dressing in the negative pressure treatment of wounds.

The invention relates to an device for the negative pressure treatment of wounds, comprising (a) a covering material for providing a wound area with an air-tight seal; (b) optionally means for connecting a negative pressure source; and (c) a wound dressing containing (c1) an open-celled foamed material, (c2) an ointment base and (c3) a swelling material, and also a process for producing a corresponding wound dressing. In addition, the invention relates to the use of an open-celled foamed material, which is wetted with an ointment base and swelling material, for application as a wound dressing in the negative pressure treatment of wounds.

A wound is understood to mean a break in the continuity of tissues in the body shell in human beings or animals. It may involve a loss of substance.

Devices for the negative pressure treatment of wounds are known in the state of the art.

WO 93/009727 A1, for example, describes a device for promoting wound healing by the application a negative pressure to the skin area containing the wound and surrounding the wound. The device according to WO 93/009727 A1 comprises a negative pressure means for generating the negative pressure, an air-tight cover for the wound, which is functionally connected to the negative pressure means, and a wound dressing for positioning on the wound inside the air-tight cover.

Apparatuses for the negative pressure treatment of wounds are commercially available, such as the VivanoTec® ex Paul Hartmann AG. In commercially available devices, a wound dressing is often used which contains an open-celled polymeric foamed material, such as polyvinyl alcohol (PVA) or polyurethane (PU), for example.

The standard commercial foam pads are compressed to different degrees, depending on the negative pressure applied. This can result in a constriction of the passageways needed for removing the exudate from the wound. In addition, it may happen that the foam may adhere to the base of the wound. Newly formed tissue may grow into the foam. This problem is a known complication in the negative pressure treatment of wounds (FDA complaint data base). In order to solve this problem, additional wound contact layers are frequently introduced between the foam and base of the wound, such as a film (see, for example, WO2001/85248). However, these additional wound contact layers reduce the passage of exudate.

Furthermore, when the dressing is changed, stuck and matted foam has to be removed in a time-consuming process, such as by rinsing with Ringer's solution. Tissue that has grown into the foam can lead to tissue traumatisation when the dressing is changed, and may thus disturb the wound healing process.

A further point is that when conventional wound dressings are used, foam particles may penetrate the wound. This problem is aggravated if the wound dressing is trimmed before use, to the size of the wound, because then in particular loose foam particles are created at the edges of the cuts. It has also been found that the standard polymeric foamed materials in some cases stick to the covering film. When the wound dressing sticks to the covering film, this is disadvantageous, especially when the dressing is removed.

The present invention is based on the problem of further improving the treatment of wounds with negative pressure and overcoming the disadvantages of the state of the art. The present invention is intended to provide devices and processes for the treatment of wounds with negative pressure, with which the treatment can be performed as effectively and gently as possible.

In particular, the present invention is intended to enable the treatment of wounds with negative pressure, which makes it possible to change a patient's wound dressings at intervals of up to three days for example. During that time, the patient is intended to have the subjective impression that the wound dressing is comfortable. Pressure irritation and reddening of the skin should be avoided. When the wound dressing is changed after a period of up to 3 days, for example, as few unpleasant odours as possible should arise. The germ count in the wound dressing removed should be low.

The intention is to provide a wound dressing in which sticking to the covering film is avoided as far as possible. In addition, it is intended that the suction force should not decline depending on the distance from the port, because of the wound dressing.

It has unexpectedly been found that the problems could be solved by using a wound dressing containing an open-celled foamed material, an ointment base and a swelling material. It has surprisingly also been found that a device for negative pressure therapy comprising at least one wound dressing wetted with an ointment base and a swelling material is particularly suitable for the advantageous, i.e. very effective and very gentle, treatment of wounds.

It has become apparent that the problem can be advantageously solved in particular if a specific foamed material and/or a specific ointment base is/are used in combination with a specific swelling material and/or if the type of foamed material and the type and quantity of the ointment base in combination with the swelling material are matched to one another.

One subject matter of the invention is therefore a device for the negative pressure treatment of wounds, comprising

(a) a covering material for sealing a wound area in an air-tight manner;

(b) optionally means for connecting a negative pressure source; and

(c) a wound dressing, containing

(c1) an open-celled foamed material,

(c2) an ointment base and

(c3) a swelling material,

wherein the total proportion of ointment base (c2) and swelling material (c3) amounts to 10 to 95% by weight, based on the total weight of the wound dressing.

A second subject matter of the invention is a process for producing a wound dressing, comprising the steps of

(I) heating an ointment base, preferably to above the dropping point;

(II) mixing swelling material and ointment base

(III) introducing an open-celled foamed material into the mixture of heated ointment base and swelling material, so that the foamed material is wetted with the mixture of ointment base and swelling material;

(IV) optionally temporarily compressing the foamed material, preferably to at least 90% and no more than 10% of its original volume, in order to achieve complete wetting of the inner surface of the foamed material with the mixture of ointment base and swelling material; and

(V) optionally removing the surplus mixture from the ointment base and swelling material, preferably by squeezing out the foamed material.

A third subject matter of the invention is the use of an open-celled foamed material (c1), which is wetted with an ointment base (2) and a swelling material (c3), as a wound dressing (c) for or in the negative pressure treatment of wounds, especially weakly exuding wounds.

The novel device in accordance with the invention, or the use of the wound dressing in accordance with the invention is characterised by a number of unexpected advantages.

As a result of wetting the foamed material with the ointment base and the swelling material, it was possible advantageously to reduce the number of particles undesirably entering the wound.

The use of the wound dressing of the invention improved the atraumatic properties, so that negative pressure treatment without any additional wound contact layers became possible.

The at least partial wetting of the foamed material with the ointment base and the swelling material reduced the hardness of the foamed material subjectively felt by the patient during the negative pressure therapy. The foamed material felt more comfortable, and patient compliance (adherence to the therapy instructions by the patient) was improved.

In addition, it has been shown that despite the use of the ointment base and the swelling material, the suction force does not decline undesirably depending on the port. It has surprisingly been found that with the wound dressing of the invention, it is possible to achieve both a sufficient transport of wound exudate and also an effect supporting wound healing, especially when an ointment base is used which comprises triglycerides and optionally diglycerides. This might be due to the activity of the lipoprotein lipase endogenously present in the wound. The fatty acids released from the triglycerides in the ointment base appear to have a positive effect on wound healing.

An effect supporting wound healing can be achieved with the wound dressing of the invention especially when the wound dressing provides the wound with a sufficient amount of ointment base and swelling material. Too large an amount of these components may, however, lead to clogging of the pores of the open-celled foamed material and thus hinder the passage of wound exudate The joint amount of ointment base and swelling material is preferably 10 to 95% by weight, more preferably 30 to 92% by weight, even more preferably 45 to 90% by weight, particularly preferably 55 to 88% by weight, especially 65 to 85% by weight, based on the total weight of the wound dressing.

The manufacturing process of the invention is technically simpler and can also be used with medical foamed materials obtained as a finished product. Wetting is performed, as a result of which the release of particles is reduced in the majority of the areas of foamed material when the foamed material is cut. In contrast to the process known from the state of the art, in which the impregnation means is added before the foam cures or is synthesised (cf. EP 0 335 669), no undesirable interactions occur between the ointment base and the swelling material, on the one hand, and the foamed material on the other hand, during curing or synthesis.

A further advantage of the wound dressing of the invention consists in the fact that the base of the wound can largely be prevented from becoming matted and/or sticking to the wound dressing itself over a period of, for example, up to 3 days. Traumatisation of the wound when changing dressings can be avoided. This enhances the efficacy of the wound treatment. This makes it possible to change a patient's wound dressings at intervals of up to three days, for example. During the period of three days, the wound dressing of the invention was felt by the patient to be comfortable. Pressure and reddening of the skin were avoided in most cases. When the wound dressing was changed after a period of 3 days, there was little unpleasant odour. The germ count in the changed wound dressing was unexpectedly low.

Another advantageous effect of the wound dressing of the invention consists in the fact that the wound does not dry out in the case of negative pressure treatment, so that even if there is little exudation, a uniformly moist environment is obtained, which is beneficial for the healing process. There is thus an unexpected hydroactive effect.

Components (a) to (c) of the device of the invention will be described below.

The device of the invention comprises a covering material (a) providing a wound area with an air-tight seal. The wound area means the wound and optionally the adjacent region around the wound. An “air-tight seal” here is not understood as implying that no exchange of gas between the wound area and its surroundings occurs. Rather, “air-tight seal” in this connection means that taking into account the negative pressure pump used, the negative pressure needed for the negative pressure treatment of wounds can be maintained. It is therefore also possible to use covering materials which exhibit a slight gas permeability, provided that the negative pressure needed for the negative pressure treatment of wounds can be maintained.

In a preferred embodiment of the invention, the covering material for providing the wound with an air-tight seal comprises a water-insoluble polymer or a metal film. The covering material is preferably from 10 μm to 10,000 μm, especially from 25 μm to 100 μm, thick.

In a preferred embodiment of the invention, the covering material (a) is a water-insoluble polymer. The water-insoluble polymer preferably has a solubility of 10 mg/l or less, more preferably 1 mg/ml or less, especially from 0.0001 to 1 mg/ml (determined using the column elution method in accordance with EU Directive RL67-548-EEC, Annex V chap. A6). Examples are polyurethane, polyester, polypropylene, polyethylene, polyamide or polyvinyl chloride, polyorganosiloxane (silicone) or a mixture thereof. The polymers mentioned are preferably present here in non-cellular form.

It has been found that a covering material with a specific water vapour permeability is able to solve the problems referred to at the beginning in a particularly advantageous manner. In a preferred embodiment, the covering material therefore has a water vapour permeability of 100 to 2,500 g/m²×24 h, more preferably from 500 to 2,000 g/m²×24 h, even more preferably from 800 to 1,600 g/m²×24 h, especially from 1,050 to 1,450 g/m²×24 h, determined in accordance with DIN EN 13726-2 at 23° C. and 85% relative humidity. Especially the combination of a covering film (a) with the above-mentioned water vapour permeability and an open-celled foamed material (c1) with the physical properties described below is particularly advantageous.

In a preferred embodiment, the covering material and the means for connecting a negative pressure source are already provided joined together ready for use. It is most particularly preferable that this embodiment should contain a film consisting of one or more water-insoluble polymers which has a self-adhesive edge, because this arrangement facilitates applying the dressing considerably.

The negative pressure therapy device of the invention optionally comprises means (b) for connecting a negative pressure source, i.e. means for producing negative pressure in the wound area. In a preferred embodiment, this is a means (b) for functionally connecting the wound area to a negative pressure source located outside the covering material, so that negative pressure can be created in the wound area and fluids can be withdrawn from the wound area.

The expression “negative pressure in the wound area” refers in connection with the invention to an air pressure within the wound dressing which is lower than the ambient air pressure (atmospheric air pressure). The expression “within the wound dressing” means the space formed between the covering material and the wound.

The pressure difference between the air pressure within the wound dressing and the ambient air pressure is expressed in the context of the invention in mm Hg (millimetres of mercury), because that is conventional in the field of negative pressure therapy. 1 mm Hg corresponds to one Ton or 133.322 Pa (Pascal). In the context of the invention, the negative pressure, i.e. the pressure difference between the air pressure within the wound dressing and the ambient air pressure is expressed as a positive figure in mm Hg.

In one embodiment of the invention, the negative pressure is a negative pressure of at least 20 mm Hg and a maximum of 250 mm Hg, preferably at least 50 mm Hg and a maximum of 150 mm Hg. This negative pressure range has proven advantageous for wound healing. In a preferred embodiment of the invention, the negative pressure is a negative pressure of at least 80 mm Hg and a maximum of 140 mm Hg, more preferably at least 120 mm Hg and a maximum of 130 mm Hg.

As explained above, the device of the invention for the negative pressure treatment of wounds preferably comprises means (b) for connecting a negative pressure source, i.e. means for functionally connecting the wound area to a negative pressure source located outside the covering material.

The functional connection may, for example, be made with a connection line or a negative pressure connector. The person skilled in the art is familiar with negative pressure connectors, which he refers to as “ports”.

In one embodiment, the means (b) is a connection line, preferably a tube, especially a silicone drainage tube. The connection line may be guided through the covering material. Alternatively, the at least one connection line may be guided beneath the edge of the covering material. In both cases, the site where it passes through must be sealed in an air-tight manner, so that the desired negative pressure in the dressing can be maintained. Suitable sealants are, for example, an adhesive film, an adhesive mixture, or an adhesive strip. The connection line may, for example, be a hose, a tube or some other body with a cavity.

In a further preferred embodiment, the means (b) is a negative pressure connector (port) which can be attached to one of the inside or outside surfaces of the covering material, with the covering material having suitable apertures for that purpose. In this embodiment too, it must be ensured that there is an air-tight seal either through the passage aperture (port inside) or the area where it is attached (port outside). The seal may, for example, be created with an adhesive film, an adhesive mixture, or an adhesive strip. It is also conceivable that the port itself may possess corresponding attachment means, such as adhesive surfaces. Suitable negative pressure connectors are commercially available. Typically, these are negative pressure connectors which are attached to the outside of the covering material. It is also convenient for the negative pressure connector to have a negative pressure adapter so that it can be connected to the other components of the negative pressure system.

When the negative pressure is applied, a suction force is achieved with which the wound exudate is extracted from the wound. The negative pressure responsible for the suction force should not decline in a disadvantageous manner depending on the distance from the port. For this reason, the difference between the “negative pressure at the port” and the “negative pressure at the wound” in the device of the invention is less than 20 mm Hg, preferably less than 15 mm Hg, more preferably less than 10 mm Hg, especially 3-8 mm Hg. This small (negative) pressure difference means that by adjusting the negative pressure at the port, it is also possible to adjust the negative pressure in the region of the wound very accurately. In addition to the above-mentioned components (a) and optionally (b), the device of the invention also comprises (c). The wound dressing (c) which is used in the device of the invention will be described in greater detail below. All the explanations concerning the wound dressing (c), including the open-celled foamed material (c1), the ointment bases (c2) and the swelling material (c3), relate not only to the device of the invention, but also to the process of the invention for producing the wound dressing and the use of the wound dressing in negative pressure therapy in accordance with the invention.

The wound dressing (c) contains an open-celled foamed material (c1), an ointment base (c2) and a swelling material (c3).

Foamed materials are normally understood to mean materials with cells (open, closed or both) distributed throughout the entire mass. Materials of this kind normally have a bulk density (according to DIN EN ISO 845) which is lower than the density of the skeleton substance.

A cell is the individual cavity formed during the production of foamed materials, which is partially or completely enclosed by cell walls and/or cell struts.

A closed cell is usually a cell which is completely enclosed by its walls and therefore does not communicate with other cells via the gas phase. An open cell is usually a cell which communicates with other cells via the gas phase. In the context of this application, the expression “open-celled” means that in the foamed material (c1), there are at least 60% open cells, preferably at least 90% open cells, more preferably at least 98% open cells, especially substantially 100% open cells, based on the total number of cells. The open-celled nature of the polyurethane foam is normally determined in accordance with ASTM D 2856-87, method B).

A cell wall is usually understood to mean the wall enclosing the cell. The cell wall may also be referred to as the cell membrane. The cell strut is usually understood to mean the region of the cell wall which separates more than two cells. Cell struts are preferably at least 1.5 times as thick, more preferably at least twice as thick, as the other regions of the cell wall.

The open-celled foamed material (c1) can preferably be a reticulated foamed material. A reticulated foamed material is understood to mean a foamed material which substantially has only cell struts. In a reticulated foamed material, the cell walls have thus been substantially removed. Reticulation is usually performed in a pressure chamber, e.g. a steel chamber. After the foamed material has been introduced into the steel chamber, the air is extracted (preferably 50 to 100% by weight of it, more preferably 70 to 99% by weight) and replaced by a combustion gas mixture, preferably a mixture containing hydrogen and oxygen, especially in a molar ratio of 2:1. When the gas mixture is ignited, the cell membranes tear because of the resulting heat and pressure wave. Where appropriate, the cell struts may also be at least partially fused, so that they are reinforced.

The open-celled foamed material (c1) preferably has a cell count (=number of pores along a straight line per running cm) of 5 to 25 cm⁻¹, more preferably 7 to 18 cm⁻¹, even more preferably 8 to 15 cm⁻¹. The cell count is preferably determined microscopically.

In principle, the open-celled foamed material (c1) can consist of any material. It should, however, satisfy certain physical requirements, because it has been found that the problems described above can be solved in an unexpectedly advantageous manner if the open-celled foamed material (c1) possesses a specific tensile strength, a specific elongation at break and/or a specific hardness. In a preferred embodiment, the open-celled foamed material (c1) has a tensile strength of 60 kPa to 500 kPa, more preferably 80 kPa to 300 kPa, even more preferably 100 kPa to 250 kPa, measured in accordance with DIN 53571 (test specimen A, pretreatment in a standardised climate). In addition, the foamed material (c1) preferably has an elongation at break of 80% to 700%, more preferably 100% to 650%, even more preferably 120% to 400%, especially 150 to 320%, measured in accordance with DIN 53571 (method 1, test specimen A). In a further embodiment, the open-celled foamed material preferably has a tensile strength of 75 to 300 kPa and an elongation at break of 100 to 350%. In addition, the foamed material (c1) preferably has a hardness of 20 to 70 Shore A, more preferably 30 to 60 Shore A, even more preferably 40 to 50 Shore A, measured in accordance with DIN 53505, the measurement being performed at 23° C. on a plate-shaped, flat and smooth test specimen 6 mm thick.

It has also been found that the problems described above can be solved in an unexpectedly advantageous manner if the open-celled foamed material (c1) possesses a specific air permeability. In a preferred embodiment, the foamed material (c1) has an air permeability of 2,500 to 10,000 l/(m²sec), more preferably 3,500 to 9,000 l/(m²sec), even more preferably 4,000 to 8,500 l/(m²sec), especially 4,500 to 8,000 l/(m²sec), measured in accordance with DIN EN ISO 9237 (20 mm testing thickness, 20 cm² testing area, 200 Pa differential pressure). Alternatively, it is a polyester-polyurethane foam with a preferred air permeability of 2,000 to 4,000 1/(m²sec), likewise measured in accordance with the above-mentioned DIN EN ISO 9237.

In particular, the combination of an open-celled foamed material (c1) with the air permeability specified above and the swelling material (c3) specified below has proven especially advantageous, e.g. with regard to wearing comfort and wound healing.

It has also been found that the problems described above can be solved in an unexpectedly advantageous manner if the foamed material (c1) exhibits viscoelastic behaviour. This is understood to mean that the behaviour of the foamed material (c) under strain looks like a combination of an elastic solid and a viscous fluid. The viscoelastic behaviour can be characterised by a torsion pendulum test in accordance with DIN 53445, method A,. It is preferable that when being analysed in accordance with DIN 53445, method A, at 23° C., the foamed material should exhibit a mechanical loss factor of 0.1 to 1.0, more preferably 0.15 to 0.8, even more preferably 0.2 to 0.6.

In a preferred embodiment, the open-celled foamed material (c1) is selected from polyurethane foams (PUR) and (preferably cross-linked) polysiloxane foams (SIL). Polyurethane foams are particularly preferred.

It has also been found that the problems described above can be solved in an unexpectedly advantageous manner if the foamed material (c1), especially a polyurethane foam, has a bulk density of between 15 and 55 kg/m³, more preferably between 18 and 40 kg/m³, even more preferably between 20 and 35 kg/m³, especially between 21 and 29 kg/m³, measured in accordance with DIN EN ISO 845 (test specimen of dimensions 100 mm×100 mm×50 mm, the measurement being performed in a standardised climate, i.e. at 23° C. and 50% relative humidity, and the test specimen being conditioned to the standardised climate for 24 h before the measurement).

In preferred embodiments of the open-celled foamed materials which can be used, they are polyurethane foams (c1-PUR) which are obtainable by reacting a curable mixture comprising the components

(i-PUR) polyisocyanate,

(ii-PUR) compounds which are reactive vis-à-vis isocyanate, such as polyol, especially polyester polyol,

(iii-PUR) catalyst,

(iv-PUR) blowing agent and

(v-PUR) optionally additives.

Isocyanates (i-PUR) that can possibly be used are generally known aliphatic, cycloaliphatic and/or especially aromatic polyisocyanates. Examples of suitable compounds for producing the polyurethanes are diphenyl methane diisocyanate (MDI), in this case especially 4,4′-diphenyl methane diisocyanate (4,4′-MDI), mixtures of monomeric diphenyl methane diisocyanates and polynuclear homologues of diphenyl methane diisocyanate (polymer-MDI), tetramethylene diisocyanate (TMDI), hexamethylene diisocyanate (HDI), toluylene diisocyanate (TDI) or mixtures thereof.

Preferably, MDI, especially 4,4′-MDI and/or HDI, is used. The 4,4′-MDI which is particularly preferably used may contain small amounts, up to about 10% by weight, of allophanate-modified or uretonimine-modified polyisocyanates. Small quantities of polyphenylene polymethylene polyisocyanate (PMDI) may also be used. The total amount of this PMDI should not exceed 5% by weight of the isocyanate.

The polyisocyanate component (i-PUR) is preferably used in the form of polyisocyanate prepolymers. These polyisocyanate prepolymers are obtainable by reacting the polyisocyanates described above (i-PUR), e.g. at temperatures of 30 to 100° C., preferably at about 80° C., with a shortfall of the polyols described below (ii-PUR) to form the prepolymer. The ratio of polyol to polyisocyanate here is selected such that the NCO content of the prepolymer is 8 to 28% by weight, preferably 14 to 26% by weight, particularly preferably 17 to 23% by weight.

Polyols such as polyetherols and/or polyesterols are commonly used as compounds (ii-PUR) which are reactive vis-à-vis isocyanates.

Polyether polyalcohols are possible (referred to in this application as “polyether polyols”) with an OH functionality of 1.9 to 8.0, an hydroxyl number of 50 to 1,000 mg KOH/g and optionally 10 to 100% primary hydroxyl groups. Polyether polyols of this kind are known, are commercially available, and are based on, for example, starter compounds which are reacted with alkylene oxides, such as propylene oxide and/or ethylene oxide, under generally known conditions. The content of primary hydroxyl groups can be reached by reacting the polyols with ethylene oxide at the end. In the production of the open-celled foamed material (c1), it is preferable for no polyether polyols to be used.

In the production of the open-celled foamed material (c1), it is preferable for polyester polyols to be used in the component (ii-PUR). The polyester polyols used (ii-PUR) are generally produced by the condensation of polyfunctional alcohols, preferably diols with 2 to 12 carbon atoms, preferably 2 to 6 carbon atoms, with polyfunctional carboxylic acids with 2 to 12 carbon atoms, such as succinic acid, glutaric acid, adipic acid, phthalic acid, isophthalic acid, and/or terephthalic acid and mixtures thereof. Examples of suitable dihydric and polyhydric alcohols are ethane diol, diethylene glycol, 1,4-butane diol, 1,5-pentane diol, and/or 1,6-hexane diol and mixtures thereof.

The reaction conditions for carboxylic acid and alcohol are usually selected such that the resulting polyester polyols do not contain any free acid groups. In addition, the resulting polyester polyols generally have a weight-average molecular weight (determined by means of gel permeation chromatography) of 500 to 3,000 g/mol, preferably more than 1,000 g/mol to 2,500 g/mol. The polyester polyols used generally have an average theoretical functionality of 2.0 to 4, preferably more than 2 to less than 3. Furthermore, the polyester polyols used generally have an average OH number of 20 to 200, preferably 30 to 90.

In a preferred embodiment, the polyester polyols used have a viscosity of 150 mPa·s to 600 mPa·s, preferably 200 mPa·s to 550 mPa·s, more preferably 220 mPa·s to 500 mPa·s, particularly preferably 250 mPa·s to 450 mPa·s and especially 270 mPa·s to 350 mPa·s, measured in accordance with DIN 53 015 at 75° C.

The compounds (ii-PUR) can be used mixed with chain extenders and/or cross-linking agents. The chain extenders are in most cases 2-functional alcohols with molecular weights of 60 to 499, such as ethylene glycol, propylene glycol, butane diol-1,4, pentane diol-1,5, dipropylene glycol and/or tripropylene glycol. The cross-linking agents are compounds with molecular weights of 60 to 499 and 3 or more active H atoms, preferably amines, and particularly preferably alcohols, such as glycerine, trimethylol propane and/or pentaerythritol.

In a preferred embodiment, the component (ii-PUR) contains (or consists of) 0-25% by weight, preferably 1 to 20% by weight, chain extender and/or cross-linking agent and 75 to 100% by weight, preferably 80 to 99% by weight polyol(s), especially polyester polyol(s), based on the total weight of the component (ii-PUR).

Standard compounds which accelerate the reaction of the component (i-PUR) with the component (ii-PUR) can be used as catalysts (iii-PUR). Conceivable examples are, for example, tertiary amines and/or organometal compounds, especially tin compounds. The following compounds may, for example, be used as catalysts: triethylene diamine, aminoalkyl and/or aminophenyl imidazoles and/or tin (ii) salts of organic carboxylic acids. Catalysts are generally used in an amount of 0.1 to 5% by weight based on the weight of the component (ii-PUR).

Generally known compounds with a chemical or physical mode of action can be used as blowing agents (iv-PUR). Water can preferably be used as a blowing agent with a physical mode of action, which forms carbon dioxide as a result of the reaction with the isocyanate groups. Examples of blowing agents with a physical mode of action are (cyclo) aliphatic hydrocarbons, preferably those with 4 to 8, particularly preferably 4 to 6 and especially 5 carbon atoms, partially halogenated hydrocarbons or ethers, ketones or acetates. The amount of blowing agents used depends on the desired density of the foamed materials. The different blowing agents may be used individually or in any combined mixtures. It is particularly preferable for only water to be used as the blowing agent, generally in an amount of 0.1 to 5% by weight, especially 2.5 to 4% by weight, based on the weight of the component (ii-PUR). Physical blowing agents are preferably used in an amount of <0.5% by weight, based on the weight of the component (ii-PUR).

The reaction optionally takes place in the presence of auxiliary substances and/or additives (v-PUR), such as fillers, cell regulators, cell openers, surface-active compounds and/or stabilisers against oxidative, thermal or microbial degradation or ageing.

As a rule, for the production of polyurethane foams, the components (i-PUR) and (ii-PUR) are reacted in such quantities that the equivalence ratio of NCO groups to the total of reactive hydrogen atoms is 1:0.8 to 1:1.25, preferably 1:0.9 to 1:1.15. A ratio of 1:1 corresponds in this case to an NCO index of 100. The desired open-celled nature of the polyurethane foam is generally ensured by an appropriate choice, with which the person skilled in the art is familiar, of the components (i-PUR) to (v-PUR). After curing, the resulting PUR foamed material is preferably reticulated. In this connection, reference is first made to the explanations provided above

Preferred embodiments of the cross-linked polysiloxane foams (c1-SIL) which can preferably be used are explained below.

According to a further embodiment, the open-celled foamed material is a polysiloxane foamed material. In general, polysiloxanes are understood to mean synthetic polymers in which silicon atoms are linked via oxygen atoms.

In a preferred embodiment, the foamed material (c1-SIL) is obtainable by reacting a curable mixture comprising the components:

(i-SIL) a polyorganosiloxane containing one or more groups with a C₂-C₆ alchemy group, preferably containing one or more vinyl groups,

(ii-SIL) a polyorganosiloxane containing one or more Si—H groups,

(iii-SIL) an organometallic catalyst and

(iv-SIL) a blowing agent containing one or more OH groups.

It has also been found that the problems described above can be solved in an unexpectedly advantageous manner if the polysiloxane foamed material (c1-SIL) has a bulk density between 0.10 and 0.35 g/cm³, more preferably between 0.12 and 0.30 g/cm³, especially between 0.15 and 0.25 g/cm³, measured in accordance with DIN EN ISO 845.

It has further been found that the problems described above can be solved in an unexpectedly advantageous manner if the open-celled foamed material (c1) contains silver in the form of silver ion or in the form of atomic silver. After the production of the open-celled foamed material (c1), it is preferable for a silver coating to be applied. Alternatively, the silver can already be incorporated in the curable composition. The foamed material (c1) preferably contains 0.000001 to 0.1% by weight, more preferably 0.0001 to 0.01% by weight silver, based on the total weight of the foamed material (c1).

In a preferred embodiment of the invention, the open-celled foamed material (c1) is 1 to 50 mm, especially 10 mm to 35 mm thick.

In a preferred embodiment, the open-celled foamed material (c1) is wetted in the dry state with the ointment base (c2) and swelling material (c3). This means that the foamed material is preferably not impregnated with an activation solution (such as Ringer's solution), for example.

In principle, the ointment bases known in the art and described in Bauer, Fromming, Führer “Lehrbuch der Pharmazeutischen Technologie”, 8th edition, chapter 12.1 to 12.6, are suitable for use as the ointment base (c2). Ointment bases are accordingly understood to mean spreadable, semi-solid preparations which are in principle suitable for use on the skin or mucous membranes, but which do not (yet) contain any pharmaceutical active agents. The person skilled in the art is familiar with the production of ointment bases; reference may be made to Ph. Eur. 6.0 “Semi-solid preparations for cutaneous application”.

Ointment bases (c2) are preferably used which do not contain an aqueous phase. In addition, it is preferable to use hydrophobic, water-absorbing and/or hydrophilic ointment bases.

Ointment bases are preferably used which contain lipids.

In the context of the present invention, the term “lipids” is used as a generic term for fats, oils, waxes and the like. The terms “oil phase” and “lipid phase” are also used synonymously. Lipids differ, inter alia, in their polarity. It has already been proposed to take the interfacial tension vis-à-vis water as a measure of the polarity of a lipid, or a lipid phase. The principle here is that the polarity of the lipid phase concerned is the greater, the lower the interfacial tension is between that lipid phase and water. According to the invention, the interfacial tension is considered a possible measure of the polarity of a given oil component. The interfacial tension is the force acting at an imaginary line, one metre long, located at the interface between two phases. The physical unit for this interfacial tension is classically calculated according to the force/length ratio and is usually expressed in mN/m. It has a positive sign if it aspires to reduce the interface. In the opposite case, it has a negative sign. For the purposes of the present invention, lipids in particular are regarded as polar if their interfacial tension relative to water is less than 20 mN/m and as non-polar if their interfacial tension relative to water is especially more than 30 mN/m. Lipids with an interfacial tension relative to water from 20 to 30 mN/m are generally described as semi-polar.

In a preferred embodiment, the ointment base (c2) contains

(c2-a) one or more polar and/or semi-polar lipids,

(c2-b) one or more non-polar lipids and

(c2-c) one or more emulsifiers.

As component (c2-a), the ointment base in this embodiment preferably contains both polar and semi-polar lipids, the weight ratio of semi-polar to polar lipids more preferably being from 10:1 to 1:10. In addition, the weight ratio of polar and/or semi-polar lipids to non-polar lipids is preferably more than 1:1 especially more than 2:1 and very particularly between 3:1 and 10:1.

In the above-mentioned embodiment, the polar and/or semi-polar lipids preferably comprise fatty acid triglycerides, fatty acid diglycerides, fatty acid monoglycerides or fatty acid esters of glycerine oligomers, such as full or partial fatty acid esters of diglycerine or triglycerine and mixtures thereof. The triglycerides, diglycerides and monoglycerides may preferably comprise esters of saturated and/or unsaturated, branched and/or unbranched alkane carboxylic acids with a chain length of 8 to 24, especially 12 to 18 C atoms. The fatty acid triglycerides, fatty acid diglycerides or fatty acid monoglycerides may advantageously be selected from, for example, the group of synthetic, semisynthetic and natural fats or oils. The proportion of polar and/or semi-polar lipids in the ointment base (c2) is preferably 20-80% by weight, more preferably 35-65% by weight.

The non-polar lipids are preferably selected from the group of branched and unbranched hydrocarbons and hydrocarbon waxes, preferably waxes, vaseline, paraffin oil, polyalkylsiloxane mineral oil and polyisobutene and mixtures thereof. The proportion of non-polar lipids in the ointment base (c2) is preferably 10-35% by weight, more preferably 20-30% by weight.

In connection with the present invention, emulsifiers should generally be understood to mean those substances which exhibit a surfactant activity, so that when water is added to the ointment base, a multi-phase mixture, namely an emulsion, can form. In particular, an ointment base of the invention should comprise at least one emulsifier by which the ointment base is enabled, when water is added, to form a water-in-oil emulsion (W/O emulsion), gel-in-oil emulsion (G/O emulsion), an oil-in-water emulsion (O/W emulsion), oil-in-gel emulsion (O/G emulsion), water-in-oil-in-water emulsion, (W/O/W emulsion) gel-in-oil-in-gel emulsion (G/O/G emulsion), gel-in-oil-in water emulsion (G/O/W emulsion), water-in-oil-in-gel emulsion (G/O/W emulsion), oil-in-water-in-oil emulsion (O/W/O emulsion), or oil-in-gel-in-oil emulsion (O/G/O emulsion). Those emulsifiers are also preferred which can form an O/W or W/O emulsion or O/G or G/O emulsion and are free of ethylene or propylene glycols or ethylene-propylene glycols, i.e. they do not include any substances that contain ethylene, propylene or ethylene-propylene glycol units.

The advantage of using an emulsifier of the O/W type is that when the composition is applied in practice, the complete composition can be washed off a wound, for example, particularly easily with water.

It is also preferable for an ointment base of the invention to contain, as component (c2-c), at least one non-ionic emulsifier with an HLB value of 3 to 18, according to the definitions listed in Rompp-Lexikon Chemie (Eds. J. Falbe, M. Regitz), 10th edition, Georg Thieme Verlag Stuttgart, N.Y., (1997), page 1764. Non-ionic O/W emulsifiers with an HLB value of 10 to 15 and non-ionic W/O emulsifiers with an HLB value of 3 to 6 are particularly preferred according to the invention.

Advantageously, the emulsifier(s), especially non-ionic 0/W emulsifier(s), can be selected from the group of
fatty alcohol ethoxylates of the general formula R—O—(CH2-CH2-O)n-H or fatty alcohol propoxylates of the general formula R—O—(CH2-CH(CH3)-O)n-H, wherein R represents a branched or unbranched alkyl or alkenyl moiety and n is a number from 10 to 50,
ethoxylated or propoxylated wool wax alcohols,
polyethylene glycol ether of the general formula R—O—(CH2-CH2-O)n-R′ or polypropylene glycol ether of the general formula R—O—(CH2-CH(CH3)-O)n-R′, wherein R and R′, independently of one another, represent branched or unbranched alkyl or alkenyl moieties and n is a number from 10 to 80,
etherified fatty acid ethoxylates of the general formula R—COO—(CH2-CH2-O)n-R′ or etherified fatty acid propoxylates of the general formula R—COO—(CH2-CH (CH3)-O)n-R′, wherein R and R′, independently of one another, represent branched or unbranched alkyl or alkenyl moieties and n is a number from 10 to 80,
esterified fatty acid ethoxylates of the general formula R—COO—(CH2-CH2-O)n-C(O)—R′ or esterified fatty acid propoxylates of the general formula R—COO—(CH2-CH(CHa)-O)n-C(O)—R<1>, wherein R and R′, independently of one another, represent branched or unbranched alkyl or alkenyl moieties and n is a number from 10 to 80,
polyethylene glycol glycerine fatty acid esters or polypropylene glycol glycerine fatty acid esters of saturated and/or unsaturated, branched and/or unbranched fatty acids and a degree of ethoxylation or a degree of propoxylation between 3 and 50,
ethoxylated or propoxylated sorbitan esters with a degree of ethoxylation or a degree of propoxylation from 3 to 100,
ethoxylated or propoxylated triglycerides with a degree of ethoxylation or a degree of propoxylation between 3 and 150,
polyoxyethylene sorbitol fatty acid esters, based on branched or unbranched alkanoic or alkenoic acids and having a degree of ethoxylation of 5 to 100, such as of the sorbet type.

Non-ionic W/O emulsifiers from the group of dicarboxylic acid esters or tricarboxylic acid esters are preferably selected as emulsifiers. Of these, esters of malonic acid, succinic acid and adipic acid in particular are used. Of these again, esters of dicarboxylic acids are preferred, especially esters of succinic acid, which are formed with saturated or unsaturated and/or linear or branched C8-C24 fatty alcohols and/or glycerine and its oligomers, especially diglycerine or triglycerine. In particular, esters of succinic acid with saturated and branched C8-C24 fatty alcohols and/or glycerine and its oligomers, especially diglycerine or triglycerine, have proven particularly advantageous as non-ionic W/O emulsifiers. Of these, dicarboxylic acid esters are most particularly suitable which are formed from succinic acid and saturated and branched C₈-C₂₄ fatty alcohols and diglycerine. One emulsifier of this kind is referred to according to the INCI nomenclature as isostearyl digyceryl succinate and is available under the product name “Imwitor(R)780”. These emulsifiers have the further advantage that they are free of polyethylene glycol, which means that they do not comprise any units of ethylene glycol. In connection with the present invention, ionic O/W emulsifiers in particular can also be used as O/W emulsifiers. As O/W emulsifiers, especially ionic O/W emulsifiers, emulsifiers can advantageously be selected from the group of esters of monoglycerides and/or diglycerides of saturated or unsaturated fatty acids with hydroxycarboxylic acids and/or tricarboxylic acids. Particularly preferable O/W emulsifiers are partially neutralised esters of monoglycerides and/or diglycerides of saturated fatty acids with hydroxycarboxylic acids and/or tricarboxylic acids, especially lactic acid and/or citric acid. Most particularly preferred are esters of lactic acid and/or citric acid, which are referred to according to the INCI nomenclature as glyceryl cocoate citrate lactate. Emulsifiers of this kind are obtainable, for example, under the product name “IMWITOR(R) 380” or “IMWITOR(R) 377”. These emulsifiers have the further advantage that they are free of polyethylene glycol, which means that they do not comprise any units of ethylene glycol.

In this context, an ointment base of the invention (c2) can preferably contain 0.5-30% by weight of at least one emulsifier (c2-c), especially 0.5-25% by weight of at least one emulsifier, especially 1-20% by weight of at least one emulsifier, especially 2.5-17.5% by weight of at least one emulsifier and most particularly preferably especially 5-15% by weight of at least one emulsifier.

In a further, alternative embodiment, hydrophobic ointment bases are used. These contain substantially no polar components and can therefore substantially not actively bind water. It is therefore a lipophilic base, in which water can only be incorporated by means of mechanical dispersion. Examples of preferred hydrophobic ointment bases are triglycerides, waxes, vaseline, paraffin oil, polyalkylsiloxane, mineral oil and polyisobutene, and mixtures thereof.

In addition, it is preferable to use water-absorbing ointment bases which contain lipophilic substances and surfactants. Examples of water-absorbing ointment bases are W/O emulsions (e.g. wool wax) or O/W emulsions.

An alternative embodiment is hydrophilic ointment bases which are miscible with water. A preferred example is a polyethylene glycol ointment base (PEG, with a weight-average molecular weight usually of 300 to 4,000 g/mol, preferably 1,500 to 3,000 g/mol).

In addition to the preferred ointment bases described above, the component (c2) also comprises cream bases (hydrophilic and hydrophobic cream bases), gels (hydrophobic gels, hydrophilic gels) and pastes (suspension ointment bases).

The ointment base (c2) ought to be semi-solid in consistency. In order to solve the problem defined at the beginning, it has proven advantageous for the ointment base to have a dropping point of 20 to 80° C., preferably 25 to 75° C., more preferably 30 to 70° C., even more preferably 35 to 65° C. and especially 40 to 60° C. The dropping point is understood in this context to mean the temperature at which the first drop of a melting substance is released from the metal nipple of a dropping point thermometer. In order to determine the dropping point experimentally, reference is made to the following explanations on FIG. 2.

In addition to the dropping point, it has proven advantageous in solving the problem described at the beginning for the ointment base (c2) to satisfy one or more of the following parameters:

acid number from 0.001 to 2.0 mg KOH/g, determined in accordance with Ph. Eur. 6.0, 2.5.1;

iodine value from 0.001 to 3.0 g 12/100 g, determined in accordance with Ph. Eur. 6.0, 2.5.4;

peroxide value from 0.001 to 1.0 mequi 0/kg, determined in accordance with Ph. Eur. 6.0, 2.5.5 A;

OH value from 1 to 100, preferably 5 to 90 mg KOH/g, determined in accordance with Ph. Eur. 6.0, 2.5.3;

saponification value from 200 to 350 mg KOH/g, preferably 240 to 300 mg KOH/g, determined in accordance with Ph. Eur. 6.0, 2.5.3; Eur 6.0, 2.5.6;

maximum heavy metal content of 10 ppm, determined in accordance with Ph. Eur. 6.0, 2.4.8.D.

Wherever nothing different is specified in the standards and Ph. Eur. regulations cited in this application, the test methods are generally performed in a standardised climate, i.e. at 23° C. and 50% relative humidity, and at an air pressure of 1013 mbar.

In a particularly preferred embodiment, triglycerides are used as the ointment base (c2).

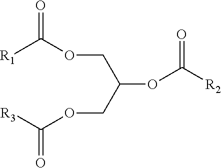

R₁, R₂ and R₃ may be the same or preferably different here.

In a preferred embodiment, the triglyceride contains a glycerine moiety and three C₆-C₂₈ acid moieties, preferably C₈-C₁₈ acid moieties. The acid moieties may be saturated or unsaturated; saturated fatty acids are preferred. The acid moieties may optionally be substituted, e.g. with a hydroxyl group.

Particularly preferably, the acid moieties contain the triglycerides of caprylic acid, capric acid, lauric acid and/or stearic acid. Triglycerides are especially preferred wherein the acid fraction contains, and especially consists of, 20 to 40% by weight caprylic acid, 10 to 30% by weight capric acid, 5 to 20% by weight lauric acid and 30 to 50% by weight stearic acid.

In an alternative particularly preferred embodiment, diglycerides are used as the ointment base (c2).

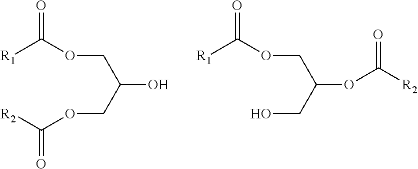

R₁ and R₂ may be the same or preferably different here.

In a preferred embodiment, the diglyceride contains a glycerine moiety and two C₄-C₂₈ acid moieties, preferably C₆-C₁₈ acid moieties. The acid moieties may be saturated or unsaturated; saturated fatty acids are preferred. The acid moieties may optionally be substituted, e.g. with a hydroxyl group. Particularly preferably, the acid moieties contain isostearic acid, stearic acid, 12-hydroxystearic acid and/or adipic acid.

In a particularly preferred embodiment, the ointment base (c2) contains a mixture of triglycerides and diglycerides. In this context, the ointment base (c2) especially contains 25 to 90% by weight, preferably 45 to 80% by weight triglycerides, and 10 to 75% by weight, preferably 20 to 55% by weight diglycerides.

Similarly, it is preferable that polyethylene glycol (PEG) is also added to the mixture of triglycerides and diglycerides. In particular, the mixture contains triglycerides/diglycerides/PEG 1 to 30% by weight, preferably 5 to 20% by weight polyethylene glycol, based on the total weight of the mixture. In this context, PEG with a weight-average molecular weight of 500 to 3,000 g/mol, especially 1,500 to 2,500 g/mol, is preferably used.

A particularly preferred ointment base (c2) contains:

20 to 90% by weight, preferably 55 to 80% by weight triglycerides, especially containing fatty acid residues, selected from caprylic acid, capric acid, lauric acid and/or stearic acid;

5 to 75% by weight, preferably 15 to 45% by weight diglycerides, especially containing fatty acid residues, selected from isostearic acid, stearic acid, 12-hydroxystearic acid and/or adipic acid; and

0 to 30% by weight, preferably 5 to 20% by weight polyethylene glycol with a weight-average molecular weight of 500 to 3,000 g/mol.

In addition to diglycerides and triglycerides, the ointment base (c2) may preferably also contain fatty alcohols, alkoxylated fatty alcohols, fatty acids and alkoxylated fatty acids.

In one possible embodiment, the ointment base (c2) does not contain any monoglycerides. In particular, the ointment base (c2) does not contain any glycerol mono-oleate.

In a preferred embodiment, the ointment base (c2) may also contain substances with an antimicrobial effect. The substance with an antimicrobial effect may, for example, be substances with amino or imino groups. In addition, the substance with an antimicrobial effect may be antimicrobially effective metal cations, especially silver cations, such as a complex of 1-vinyl-2-pyrrolidone with silver cations. Particularly suitable substances with an antimicrobial effect are also biguanide derivatives such as chlorhexidine or polybiguanides, such as polyethylene biguanide (PEB), polytetramethylene biguanide (PTMB) or polyethylene hexamethylene biguanide (PEHMB). A particularly preferred polybiguanide is polyhexamethylene biguanide (PHMB, or polyhexanide). Further suitable substances with an antimicrobial effect are polyguanidines, such as polyhexamethylene guanidine (PHMG), N-octyl-1-[10-(4-octyliminopyridine-1-yl)decyl]pyridine-4-imine (octenidine), quaternary ammonium compounds, such as benzalconiumchloride or cetylpyridinium chloride, triazines such as 1-(3-chloroallyl)-3,5,7-triaza-1-azonia-adamantan-chloride or the ammonium compound taurolidine.

Substances with an antimicrobial effect are normally contained in the ointment base (c2) in an amount of 0 to 15% by weight, preferably 0.1 to 5% by weight, based on the total weight of the ointment base (c2).

A swelling material (c3) should be understood in the context of the present invention as meaning a synthetic or natural polymeric material, preferably a hydrophilic synthetic or natural polymeric material, which is preferably capable of forming a gel in water. The swelling material capable of forming a gel can dissolve colloidally in water. Alternatively, the swelling material absorbs water and/or swells in water.

In a preferred embodiment, gums can be used as the swelling material (c3).

Gums are a heterogeneous group of biopolymers. Gums may comprise animal gums containing glycoproteins, and/or vegetable gums containing (hetero)polysaccharides.

Examples of vegetable gums are agar, alginic acid and its salts and derivatives, guar gum, chitin and its derivatives, chitosan (derivatives), carrageenan, xanthan gum, gum arabic, gum traganth, gellan gum and pectin. Mixtures thereof can likewise be used.

Examples of animal gums are gelatines, peptin and glycoproteins.

In a further preferred embodiment, cellulose and/or its derivatives can also be used as the swelling material (c3). The group of cellulose derivatives in connection with the present invention especially includes cellulose ethers and cellulose esters and their salts. As cellulose ethers in this context, hydroxyalkyl celluloses, especially hydroxy-C₁₋₆-alkyl cellulose such as hydroxymethyl cellulose, hydroxyethyl cellulose, hydroxypropyl cellulose or hydroxybutyl cellulose and most particularly preferably hydroxymethyl cellulose or hydroxyethyl cellulose, are preferably used. As cellulose esters, carboxyalkyl cellulose, especially carboxy-C₁₋₆-alkyl cellulose, such as carboxymethyl cellulose, carboxyethyl cellulose, carboxypropyl cellulose or carboxybutyl cellulose or their salts, and most particularly preferably carboxymethyl cellulose or carboxyethyl cellulose or their salts in particular, especially the sodium salt, are used. Mixtures thereof can also be employed. The number-average molecular weight of the celluloses and/or their derivatives is preferably 1,000-250,000 g/mol, more preferably 5,000-175,000 g/mol, especially 10,000-100,000 g/mol. The number-average molecular weight of the cellulose and/or its derivatives is preferably determined by means of gel permeation chromatography.

In a further preferred embodiment, the swelling material (c3) may also be a synthetic polymer. The synthetic polymer has a number-average molecular weight of 2,500 to 250,000,000 g/mol, preferably 5,000 to 5,000,000 g/mol, more preferably 50,000 to 1,000,000 g/mol. The number-average molecular weight of the synthetic polymer is preferably determined by means of gel permeation chromatography.

Synthetic polymers which are suitable as swelling material (c3) are, for example, polyvinyl alcohol, poly(meth)acrylates and polyvinyl pyrrolidone, especially crospovidone.

The swelling material (c3) may be present both in the form of fibres and also in the form of particles and/or fibres dispersed in the wound dressing (c). In particular, the swelling material (c3) may be present in the form of particles. Swelling materials (c3) present in particulate form are particularly preferred. The particles preferably have a D₅₀ value for the particle size of 1-200 μm, more preferably 5-150 μm and especially 10 to 90 μm, measured with a “Mastersizer 2,000” ex Malvern Instruments. In addition, the swelling material (c3) usually has a water content of less than 10% by weight, preferably less than 5% by weight, more preferably 0.01 to 2.5% by weight, based on the swelling material particles. The water content is determined in accordance with the Karl Fischer method, which is described in Ph. Eur. 6th edition, 2008, chapter 2.5.12. The determination step is preferably conducted using a Karl Fischer titrator, Mettler Toledo DL32. Samples of 50 to 100 mg are usually analysed.

In addition, swelling materials (c3) can preferably be used which are intermolecularly and/or intramolecularly cross-linked. These swelling materials (c3) are not soluble in water, for example, but do swell when liquid is added. Because of their inner cohesion, these swelling materials are then present in disperse form.

According to a particularly preferred embodiment, the wound dressing of the invention (c) preferably contains at least one of the above-mentioned swelling materials (c3).

According to a further preferred embodiment, the wound dressing of the invention (c) preferably contains at least two of the above-mentioned swelling materials (c3).

In a preferred embodiment of the present invention, the weight ratio of ointment base (c2) to swelling material (c3) is from 1:1 to 20:1, preferably from 2:1 to 10:1, more preferably from 3:1 to 6:1.

The open-celled foamed material (c1) is wetted with the ointment base (c2) and swelling material (c3). “Wetting” in this context is understood to mean that the ointment base covers, preferably completely covers, the outer and inner surfaces (=the inner surface formed by the open pores) of the foamed material. In a preferred embodiment, at least 20%, more preferably at least 50%, even more preferably at least 70%, especially at least 90% of the total surface area of the foamed material (c1) is covered with ointment base (c2) and swelling material (c3). Wetting of the surface of the foamed material (c1) with ointment base (c2) and swelling material (c3) can also be referred to as impregnating.

It has been found that specific amounts of ointment base (c2) and swelling material (c3) are able to solve the problems described at the beginning in a particularly advantageous manner. In a preferred embodiment, the proportion of ointment base (c2) and swelling material (c3) is 10 to 95% by weight, more preferably 30 to 92% by weight, even more preferably 45 to 90% by weight, particularly preferably 55 to 88% by weight, especially 65 to 85% by weight, based on the total weight of the wound dressing (c). The total weight of the wound dressing (c) is obtained by adding the weights of components (c1), (c2) and (c3).

As a matter of principle, all the explanations concerning preferred embodiments of individual parameters of the open-celled foamed material (c1) and/or the ointment base (c2) and the swelling material (c3) should not be seen in isolation, but also apply in combination with the explanations concerning preferred embodiments of other parameters, or in combination with the explanations concerning the compositions of substances (c1) and (c2) and (c3).

The wound dressing (c) containing (c1), (c2) and (c3) can advantageously be produced by the process of the invention. This comprises the steps of:

(I) heating an ointment base, preferably to above the dropping point;

(II) mixing swelling material and ointment base

(III) introducing an open-celled foamed material into the mixture of swelling material and heated ointment base, so that the foamed material is at least partially wetted with the mixture of ointment base and swelling material;

(IV) optionally temporarily compressing the foamed material, preferably to at least 50% and no more than 90% of its original volume, in order to achieve wetting of the inner surface of the foamed material with the mixture of ointment base and swelling material; and

(V) optionally removing the surplus mixture from the ointment base and swelling material, preferably by squeezing out the foamed material.

In step (I) the ointment base is heated, preferably to about 10 to 30° C. above the dropping point;

In step (II), the swelling material, for example, is added and the components are mixed.

In a further preferred embodiment, the addition of the swelling material and/or the mixing of the components can also occur before or during step (I).

In step (III), the foamed material is introduced into the mixture from step (II) and preferably immersed. The dwell time in the mixture from step (II) depends on the size of the foamed material and is usually 10 to 100 seconds.

Step (IV) may take place during or after, preferably during immersion.

Temporarily compressing the foamed material is understood here to mean that the foamed material is compressed manually or by a suitable machine and then released again. In this context, the foamed material can be released again immediately after compressing, or may also be held for a short time, such as 1 to 10 seconds, in the compressed state and then released again. After the foamed material has been released following the temporary compression, the foamed material can then return to its original volume either completely or largely. During the relaxing process, the mixture from step (II) is usually absorbed, whereby preferably complete wetting of the inner surface of the foamed material with the mixture from step (II) is achieved.

After that, the foamed material wetted with the mixture from step (II) is removed. Surplus mixture from step (II) can be squeezed out in step (V). Squeezing out is preferably performed before the ointment base has cooled to below the dropping point.

The squeezing force is preferably selected such that the proportion of ointment base and swelling material is 10 to 95% by weight, more preferably 30 to 92% by weight, even more preferably 45 to 90% by weight, particularly preferably 55 to 88% by weight, especially 65 to 85% by weight, based on the total weight of the wound dressing.

All the explanations provided above on preferred embodiments of components (c), (c1), (c2) and (c3) also apply to the process of the invention.

A preferred embodiment is a wound dressing which is obtained by the process of the invention.

In addition, the invention provides a ready-to-use kit for negative pressure wound treatment comprising the device of the invention, wherein the wound dressing (c) is packed ready to use.

One subject matter of the invention is thus a ready-to-use kit for negative pressure wound treatment, comprising

(a) covering material for sealing a wound area in an air-tight manner, i.e. the wound and the wound surroundings,

(b) optionally means for connecting a negative pressure source, preferably means for functionally connecting the wound area to a negative pressure source located outside the covering material, so that negative pressure can be created in the wound area and fluids can be withdrawn from the wound area. and

(c) a wound dressing packed ready to use, containing

(c1) an open-celled foamed material,

(c2) an ointment base and

(c3) a swelling material,

wherein the total proportion of ointment base (c2) and swelling material (c3) amounts to 10 to 95% by weight, based on the total weight of the wound dressing. It is preferably a triglyceride ointment base.

The packed wound dressing (c) comprised in the kit is preferably packed so as to be moisture-tight. The ready-to-use wound dressing is preferably provided in sterile form, wherein the sterilisation can be achieved with moist heat, ethylene oxide gas treatment and/or irradiation.

Sterilisation by irradiation is preferred. The irradiation sterilisation can be carried out with beta and/or gamma rays. Beta radiation is preferred. Sterilisation can be achieved by irradiation with a dose of 20 to 60 kGy (20 to 60 kJ/kg). It has been found that a dose of 25 to 40 kGy (25 to 40 kJ/kg) is preferably used. Alternatively, gamma radiation is also preferred. It has been found that with the preferred irradiation methods, an unexpectedly advantageous sterilisation of the wound dressing can be achieved.

The kit may include further optional components, such as adhesive means for fixing the dressing, sealing means for producing an air-tight seal in the dressing, pressure sensors, connection elements for pressure sensors, additional tubes, connectors for tubes, disinfectants, skin-care products, pharmaceutical preparations or instructions for use. The kit of the invention preferably also contains scissors, swabs and/or tweezers, especially in sterile form.

The kit may comprise both the at least one wound contact layer, and also at least one additional pressure distribution layer. The kit preferably also comprises a ready-to-use negative pressure unit.

A further subject matter of the invention is the use of the above-mentioned wound dressing (c) for or in the negative pressure treatment of wounds, i.e. the use of an open-celled foamed material (c1), which is wetted with an ointment base (2) and a swelling material (c3), for the negative pressure treatment of wounds, especially as a wound dressing (c). A further preferred subject matter of the invention is a wound dressing (c) containing an open-celled foamed material (c1), an ointment base (c2) and a swelling material (c3), for the negative pressure treatment of wounds. A further preferred subject matter of the invention is a process for the treatment of a wound with a wound dressing in accordance with the present application. All the explanations provided above on preferred embodiments of components (c), (c 1), (c2) and (c3) also apply both individually and in combination to the use in accordance with the invention.

For example, the invention relates to the use of a polyurethane foam (c 1) which is obtainable by reacting a mixture comprising the components

(i) polyisocyanate,

(ii) polyol, especially polyester polyol,

(iii) blowing agents, and

(iv) catalyst;

wherein the foamed material is preferably obtainable by reacting a polyisocyanate (i), selected from MDI, PMDI and/or TDI, with a (ii) polyester polyol, which is preferably obtainable by reacting a dicarboxylic acid with 4 to 8 carbon atoms with a dialcohol with 2 to 6 carbon atoms, wherein the (ii) polyester polyol preferably has a weight-average molecular weight of 500 to 4,000 g/mol;

the open-celled polyurethane foam preferably having an aromatic content of 5 to 50%, more preferably 10 to 45%, especially 15 to 40%;

the open-celled polyurethane foam preferably having an elongation at break of 80% to 700%, more preferably 100% to 650%, even more preferably 120% to 400%, especially 150 to 320%;

a mechanical loss factor preferably of 0.1 to 1.0, more preferably 0.15 to 0.8, even more preferably 0.2 to 0.6;

the open-celled polyurethane foam preferably having a hardness of 20 to 70 Shore A, more preferably 30 to 60 Shore A, even more preferably 40 to 50 Shore A;

the open-celled polyurethane foam preferably having a cell count (=number of pores along a straight line per running cm) of 3 to 40 cm⁻¹, preferably 5 to 25 cm⁻¹, more preferably 7 to 18 cm⁻¹, even more preferably 8 to 15 cm⁻¹;

the open-celled polyurethane foam preferably having a bulk density between 15 and 55 kg/m³, more preferably between 18 and 40 kg/m³, even more preferably between 20 and 35 kg/m³, especially between 21 and 29 kg/m³; and/or

the open-celled polyurethane foam preferably having an air permeability of 2,500 to 10,000 l/(m²sec), more preferably 3,500 to 9,000 l/(m²sec), even more preferably 4,000 to 8,000 l/(m²sec) and especially preferably 4,500 to 7,000 l/(m²sec). Alternatively, the open-celled polyurethane foam, especially the polyester-polyurethane foam, may have an air permeability of 2,000 to 4,000 l/(m²sec).

In the use in accordance with the invention, this polyurethane foam (c1) is wetted with an ointment base (c2) and a swelling material (c3), wherein the ointment base (c2) has a dropping point of 20 to 80° C., preferably 25 to 75° C., more preferably 30 to 70° C., even more preferably 35 to 65° C. and especially 40 to 60° C.;

is preferably selected from a hydrophobic, water-absorbing and/or hydrophilic ointment base;

preferably comprises (c2-a) one or more polar and/or semi-polar lipids;

(c2-b) one or more non-polar lipids and

(c2-c) one or more emulsifiers.

more preferably (c2-a) 20-80% by weight monoglycerides, diglycerides and/or triglycerides and/or full or partial esters of oligomers of glycerine,

(c2-b) 10-30% by weight non-polar lipids, preferably selected from the group of vaseline, petrolatum, paraffin, waxes and mixtures thereof,

(c2-c) 0.5-30% % by weight of an emulsifier, preferably an ionic O/W emulsifier or a non-ionic W/O-emulsifier;

and wherein the swelling material (c3)

is understood as meaning a synthetic or natural polymeric material, preferably a hydrophilic synthetic or natural polymeric material, which is preferably capable of forming a gel in water and wherein

the weight ratio of ointment base (c2) to swelling material (c3) is from 1:1 to 20:1, preferably from 2:1 to 10:1, more preferably from 3:1 to 6:1.

Particular advantages achieved by the device of the invention, the kit of the invention or the use or application in accordance with the invention result when the wounds are mildly exuding wounds. Mildly exuding wounds are understood to mean wounds that secrete between 0.01 and 0.4 (g/cm²)/24 h wound exudate.

In a further preferred embodiment, the wound dressing (c) (containing (c1), (c2) and (c3)) can be provided for use in negative pressure therapy in the treatment of a wound caused by a skin transplant. The application comprises the treatment of wounds caused by split skin transplants and full skin transplants, by means of negative pressure therapy. Advantageous effects result from the structure of the open-celled foamed material (c1) which is wetted with the ointment base (c2) and swelling material (c3), and from the even distribution of pressure. When the wound dressing (c) is used in the treatment of a wound resulting from a skin transplant, the skin graft can be fixed sufficiently firmly in place and at the same time harmful shear forces can be avoided.

The wound dressing (c) described above can advantageously be used in the negative pressure treatment of pressure sores in patients with a body mass index (BMI=body mass divided by body height squared) of less than 18.0, especially with a body mass index of 14 to 17.5. This is especially valid if it is a patient aged more than 60. The advantageous effect of the device of the invention or of the kit of the invention is particularly pronounced in these patients.

A further subject matter of the invention is a process for the negative pressure treatment of wounds, comprising the steps of

a) preparing an device in accordance with any of claims 1 to 12;

b) applying the negative pressure dressing to the wound;

c) producing a negative pressure of 20 mm Hg to 250 mm Hg, preferably 50 mm Hg to 150 mm Hg in the wound area for at least 30 minutes and no more than 7 days, preferably for at least 1 day and no more than 6 days.

### FIGURES

FIG. 1: Schematic structure of the device the of the invention (side view)

FIG. 2: Apparatus for determining the dropping point

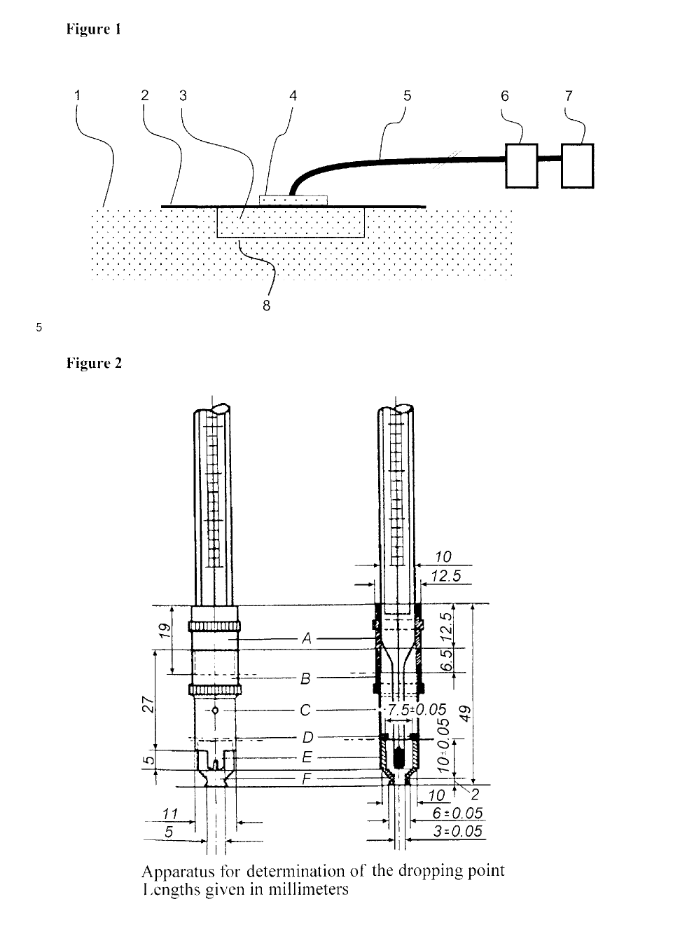

### LEGEND TO FIG. 1

1 wound surroundings (i.e. undamaged skin as a rule)

2 air-impermeable covering material (a)

3 wound dressing (c)=open-celled foamed material (c1), wetted with ointment base (c2) and swelling material (c3)

4 negative pressure connector (port)

5 negative pressure connection line

6 collection vessel

7 negative pressure unit

8 base of the wound

The device of the invention for the negative pressure treatment of wounds is explained in more detail by means of FIG. 1. The invention should not, however, be understood as being reduced to the embodiments illustrated in the drawings or in the description of the drawings. On the contrary, the device of the invention also comprises combinations of the individual features of the alternative embodiments.

FIG. 1 illustrates the schematic structure of the device of the invention in a side view. The device comprises an air-impermeable covering material (2), means (4-5) for functionally connecting the wound area to a negative pressure source (7) located outside the covering material, and the wound dressing (3), containing open-celled foamed material (c1), ointment base (c2) and swelling material (c3). The covering material (2) is attached in the area of the wound surroundings (1), where there is usually undamaged skin. The size of the covering material ought to be such that the covering material can be attached outside the wound area in the area of the wound surroundings (1). The covering material (2) can be provided in various dimensions and shapes, such as circular, oval or rectangular. It may also be provided in an irregular shape adapted to the wound. The covering material (2) is usually attached in the area of the wound surroundings (1) and sealed in an air-tight manner. This can be done, for example, by providing the covering material (2) with an adhesive edge. Alternatively, an adhesive substance may either be applied to the edge of the covering material (2) and/or to the intact skin in the area of the wound surroundings. The advantage of this is that it is easier to adapt the covering material to the shape and size of the wound. The negative pressure connector (4) in the preferred embodiment shown here is attached to the outside of the air-impermeable covering material (2), facing away from the wound. In order functionally to connect the wound area to a negative pressure unit (7) located outside the covering material, there must in this arrangement be one or more opening(s) passing through the covering material (2) in the region of the negative pressure connector (4).

In addition, an air-tight seal must be ensured. Such a seal can be created by, for example, applying a film (not shown in FIG. 1) to the top side of the port facing away from the wound and sticking it to the covering material (2). Applying the dressing can be made easier if a port is used in which a suitable fixing and sealing means for fixing the port on the covering material is already present. This is the case, for example, with the commercially available PPM-Drainageport ex Phametra-Pharma and Medica-Trading GmbH (Herne/Ruhrstadt, Germany).

In a preferred embodiment of the invention, the device for the negative pressure treatment of wounds does not comprise a wound contact layer for insertion between the wound dressing (3) and the wound surface (8).

FIG. 2 shows the apparatus for determining the dropping point. The process for determining the dropping point experimentally is performed as follows:

Test equipment:—Ubbelohde dropping point thermometer
0-110° C. calibrated
beaker 1,000 ml
test tube, approx. 200 mm long, diameter: 40 mm, with pierced bung
magnetic stirrer with heating plate
filter paper blanks 10×10 mm

Reagents:—demineralised water
apparatus for determining the dropping point in accordance with FIG. 2

The apparatus (see FIG. 2) consists of 2 metal sleeves (A) and (B) screwed together. Sleeve (A) is attached to a mercury thermometer. A metal nipple (F) is loosely attached to the lower part of sleeve (B) by 2 clamping jaws (E). Locking pins (D) 2 mm long fix the position of the nipple accurately. They likewise serve to centre the thermometer. An opening (C) in the wall of sleeve (B) allows for pressure equalisation.

The dropping surface of the nipple must be flat and the edges of the exit aperture must be at a right angle to it. The lower part of the mercury thermometer is of the shape and dimensions show in the illustration. The thermometer permits temperature measurements from 0 to 110° C., its scale division is 1° C. (1 mm each). The mercury bulb of the thermometer has a diameter of 3.5±0.2 mm and a height of 6.0±0.3 mm

The entire apparatus was hung in the middle of a test tube about 200 mm long and with an external diameter of 40 mm by means of a pierced bung, through which the thermometer was inserted. The bung had a notch at the side. The opening of the nipple must be 15 mm above the bottom of the test tube. The entire arrangement was immersed in a 1-litre beaker filled with water. The bottom of the test tube must be about 25 mm above the bottom of the beaker. The water level must reach the upper part of the sleeve (A). A stirrer ensures that the bath retains an even temperature.

The test procedure was conducted as follows.

The nipple was filled completely with the unmolten substance to be tested, unless something else is prescribed. The excess substance was scraped off both ends of the nipple with a spatula. The sleeves (A) and (B) were screwed together, and the nipple inserted into the sleeve (B) as far as the locking pins. The substance ejected by the thermometer at the nipple opening was scraped off with a spatula. As described above, the apparatus was hung in the water bath. After that, the water bath was heated up in such a way that from about 10° C. below the expected dropping point, the temperature rose by about 1° C. per minute. The temperature was read when the first drop fell off the nipple. The process was repeated at least three times with new samples; a maximum difference of 3° C. between the individual values was permitted.

Evaluation: the dropping point is deemed to be the average of three tests.

The invention will now be illustrated with reference to the following examples.

### EXAMPLES

### Example 1

### Production of a Composition of Ointment Base (c2) and Swelling Material (c3)

Ingredients:

| | |
|---|---|
| 1. Isostearyl diglyceryl succinate (non-ionic W/O | 5% by weight |
| emulsifier) | |
| 2. Glyceryl stearate (co-emulsifier) | 4% by weight |
| 3. Hydrogenated coco glycerides (polar lipid) | 4% by weight |
| 4. Caprylic/capric/myristic/stearic triglycerides (polar | 23% by weight |
| lipid) | |
| 5. Bis-dyglyceryl polyacyladipate-2 (polar lipid) | 19% by weight |
| 6. Vaseline (non-polar lipid) | 25% by weight |
| 7. Sodium caboxymethyl cellulose (swelling material) | 20% by weight |

In order to form an ointment base (c2), ingredients 1 to 6 were melted at approx. 75-80° C. while being stirred. After that, ingredient 7 was dispersed in the ointment base with vigorous stirring. The mixture was cooled with further vigorous stirring, and a fine crystal structure formed.

### Example 2a

### Production of a Wound Dressing of the Invention (c)

The composition of example 1 (dropping point approx. 50° C.) was heated to 55° C. An open-celled polyurethane foam (bulk density: 23 kg/m³, thickness: 32 mm, air permeability: 5,500 l/(m²sec)) was immersed and compressed to approx. 70% of its original volume, so that when the foam expanded back to its original volume after compression, the inner surface of the foam was also completely wetted with ointment base. After being removed, the foamed material was squeezed, so that the proportion of ointment base and swelling material after squeezing was 79% by weight.

### Example 2b

### Production of a Wound Dressing of the Invention (c)

The composition of example 1 (dropping point approx. 50° C.) was heated to 55° C. An open-celled polyurethane foam (bulk density: 23 kg/m³, thickness: 32 mm, air permeability: 6,000 l/(m²sec)) was immersed and compressed to approx. 70% of its original volume, so that when the foam expanded back to its original volume after compression, the inner surface of the foam was also completely wetted with ointment base. After being removed, the foamed material was squeezed, so that the proportion of ointment base and swelling material after squeezing was 79% by weight.

### Example 3

### Testing the Wound Dressing of Example 2a in the Wound Simulator

The wound dressing of example 2a was tested in a negative pressure wound simulator (described in DE 10 2008 064 510).

For a “well-conducted” negative pressure therapy simulation, the results of the test had to demonstrate only very small differences in pressure between the wound area (sensor 1) and the port area (sensor 2) and uniform behaviour over a lengthy period, with at the same time a linear pattern with regard to the exudates withdrawn. Any difference in pressure is due to the wound dressing. The negative pressure was generated using an ATMOS 5041 wound drainage suction unit. The exudate was created by a B. Braun Perfusor F® spray pump, which can generate a constant flow. A PPM drainage port system (Herne/Ruhrstadt, Germany) was used.

TABLE 1
Parameters

| | | | | Exudate | | |
|---|---|---|---|---|---|---|
| | | | | | Viscosity | |
| | | Pressure at | Pressure at | Flow | (mPa · s) | |
| Wound | Temperature | the port | the wound | rate | (blood substitute | Wound size |
| dressing | (° C.) | (mm Hg) | (mm Hg) | (ml · h⁻¹) | solution) | (cm²) |
| Wound dressing of the | 32 | 127 | 122 | 10 | 4.5 | 28.3 |
| invention in accordance | | | | | | |
| with example 2a | | | | | | |

The blood substitute solution was produced as follows.

425 g glycerine, 566 g demineralised water, 9 g sodium chloride and 0.2 g Allura Red were mixed together and blended for 15 minutes. The resulting blood substitute solution had a viscosity of 4.5 mPa·s.

The wound dressing was applied to the wound simulator and covered with Hydrofilm® in order to create an air-tight system. Over the artificial wound, a small hole was cut in the film, and a PPM port was attached so that the exudate could flow out of the wound area. This port was connected first to a container in order to collect the exudate, and secondly to the pressure gauge in order to measure the pressure within the port system. The container was connected to a negative pressure pump, and the amount of exudate extracted was measured by determining the weight of the exudate by means of a scale. The other pressure sensor, which was attached inside the wound simulator, measured the pressure inside the simulated wound. The experiment was conducted in accordance with the parameters shown in Table 1.

The results showed that the pressure difference during the entire experiment remained low, and the behaviour of the pressures remained normal. The exudates calculated and collected displayed a very similar curve, which also demonstrated the success of the experiment.

That leads to the following conclusion.

The amount of exudate collected increased linearly in accordance with expectations, and the pressure difference displayed very good results. This means that in accordance with example 1, the wound dressing of the invention in worked consistently and unexpectedly advantageously in an NPWT simulation process.

1. A device for the negative pressure treatment of wounds comprising,
(a) a covering material for sealing a wound area in an air-tight manner;
(b) optionally means for connecting a negative pressure source; and
(c) a wound dressing, containing
(c1) an open-celled foamed material, preferably a polyurethane foam,
(c2) an ointment base and
(c3) a swelling material,
wherein the total proportion of ointment base (c2) and swelling material (c3) amounts to 10 to 95% by weight, based on the total weight of the wound dressing.

2. The device of claim 1, wherein the open-celled foamed material (c1) has a tensile strength of 60 to 500 kPa, measured in accordance with DIN 53571 and/or an elongation at break of 80% to 700%, measured in each case in accordance with DIN 53571.

3. The device of claim 1, wherein the open-celled foamed material (c1) has an air permeability of 2,000 to 9,0001/(m²sec), measured in accordance with DIN EN ISO 9237.

4. The device of claim 1, wherein the open-celled foamed material (c1) has a bulk density between 10 and 40 kg/m³, measured in accordance with DIN EN ISO 845.

5. The device of claim 1, wherein the open-celled foamed material (c1) is a polyurethane foam obtainable by reacting a mixture comprising the components
(i) polyisocyanate,
(ii) polyol, especially polyester polyol,
(iii) catalyst and
(iv) blowing agents.

6. The device of claim 1, wherein the open-celled foamed material (c1) is a polyester-polyurethane foam with an air permeability from 2,000 to 4,000 l/(m²sec), measured in accordance with DIN EN ISO 9237 and the joint proportion of ointment base (c2) and swelling material (c3) is 33 to 71% by weight, based on the total weight of the wound dressing.

7. The device of claim 1, wherein the ointment base (c2) comprises:
(c2-a) one or more polar and/or semi-polar lipids;
(c2-b) one or more non-polar lipids; and
(c2-c) one or more emulsifiers.

8. The device of claim 1, wherein the ointment base (c2) comprises:
(c2-a) 20-80% by weight monoglycerides, diglycerides and/or triglycerides and/or full or partial esters of oligomers of glycerine,
(c2-b) 10-30% by weight non-polar lipids, preferably selected from the group of vaseline, petrolatum, paraffin, waxes and mixtures thereof, and
(c2-c) 0.5-30% % by weight of an emulsifier, preferably an ionic O/W emulsifier or a non-ionic W/O emulsifier;

9. The device of claim 1, wherein the ointment base (c2) contains a triglyceride, wherein it is preferably a triglyceride containing a glycerine moiety and three C₆-C₂₈ acid moieties, preferably C₈-C₁₈ acid moieties.

10. The device of claim 1, wherein the ointment base (c2) contains a diglyceride, wherein it is preferably a diglyceride containing a glycerine moiety and two C₆-C₂₈ acid moieties, preferably C₈-C₁₈ acid moieties.

11. The device of claim 1, wherein the ointment base (c2) has a dropping point if 20 to 80° C.

12. The device of claim 1, wherein the weight ratio of ointment base (c2) to swelling material (c3) is 1:1 to 20:1, preferably 2:1 to 10:1, more preferably 3:1 to 6:1.

13. A process for producing a wound dressing, comprising the steps of
(I) heating an ointment base, preferably to above the dropping point;
(II) mixing swelling material and ointment base;
(III) introducing an open-celled foamed material into the mixture of swelling material and heated ointment base, so that the foamed material is at least partially wetted with the mixture of ointment base and swelling material;
(IV) optionally temporarily compressing the foamed material, preferably to at least 50% and no more than 90% of its original volume, in order to achieve wetting of the inner surface of the foamed material with the mixture of ointment base and swelling material; and
(V) optionally removing the surplus mixture from the ointment base and swelling material, preferably by squeezing out the foamed material.

14. A wound dressing obtainable by a process as claimed in claim 13.

15. An open-celled foamed material which is wetted with a triglyceride ointment base and a swelling material, for use as a wound dressing for the negative pressure treatment of wounds, which are preferably mildly exuding wounds.
